(19) United States
(12) Patent Application Publication
(10) Pub. No.: US 2026/0000305 A1
(43) Pub. Date: Jan. 1, 2026

(54) AIR Blood pressures

(71) Applicant: Anuthep BENJA-ATHON, Manhasset, NY (US)

(72) Inventor: Anuthep BENJA-ATHON, Manhasset, NY (US)

(21) Appl. No.: 18/831,085

(22) Filed: Jun. 27, 2024

(51) Int. Cl. A61B 5/021; G16H 20/10

(52) U.S. Cl. CPC A61B 5/02108; G16H 20/10

(57) ABSTRACT

Individual interactive and adaptable digital comparative graphs of synchronized and controlled normotensive SBP and DBP created and controlled by individual antihypertensive medications therapies of therapeutic potencies, which decreasing and preventing morbidity and mortality, in the users and wears of the mobile digital blood pressure monitor devices.

### FIELD OF THE INVENTION

The abiotic intelligence-rendered digital management of hypertension

### BACKGROUND OF THE INVENTION

The leading cause of death in USA is the cardiovascular diseases. And hypertension is one of the major causes of cardiovascular diseases.

About 80 million Americans, over 1 billion worldwide and 1.5 billion by 2025 have hypertension. Hypertension is more prevalent in developing nations due to poor treatment and control . . . (Systemic hypertension: Mechanisms and diagnosis. Braunwald's Heart Disease. 11th ed. 2019 Vol 1 page 910)

AIR Blood Pressures is the most effective treatment of hypertension in billions of people in the history of mankind. The present invention—the abiotic intelligence-rendered blood pressures (AIR Blood Pressures)—is the one and only technology for most effectively managing hypertension—unbeknown to physicians, healthcare providers and patients—which may explain the aforementioned preamble.

The impossibilities in the treatments of hypertension and lack of the effective therapies in billions of people:

The immense cardiovascular system of dynamic cardiovascular hemodynamics equiped with ubiquitous evolution superbly engineered neurological and chemo sensors and regulators.

There are only about 100,000 physicians of about 85 medical specialties in USA. Thus, it is impossible for the present conventional method and system to effectively managing patients in the erstwhile measurement in the clinics visits, erstwhile measurement offices visits, infrequent homes usage of BP cuffs (for lack of reasons) trusting the prescribed medications and any combinations thereof.

Furthermore, the immense permutations and diverse circumstances: there are 14 pharmaceutical classes comprising about 95 different antihypertensive medications each of which has different dosages including daily, twice a day, three times a day and four times a day; and fluctuating SBP and DBP and HR throughout the days in people of diverse ethnicities, circumstances and metabolisms, drug bioavailability and interactions. Thus, large permutations of drugs in diagnosed hypertensive patients most of whom either lacking or not accessing to information, do not effectively know how to and when to use the medications and what the readings or the significance are either uninformed, poorly informed and noncompliant patients. In brief, the aforementioned posing immense, very common and life-and-death health and healthcare problems.

### SUMMARY OF THE INVENTION

It is most likely true that most, if not all, hypertensive people do not know the values of their systolic and diastolic blood pressures at any given time.

The AIR Blood Pressures creating individual interactive and adaptable digital comparative graphs of synchronized and controlled normotensive SBP and DBP created and controlled by individual medical prescribed dosages of antihypertensive medications of therapeutic potencies ingested by the users and wears of the mobile digital blood pressure monitor devices, which created and sharing the SBP and DBP in and among networked communication devices, thereby decreasing the risks of and preventing cardiovascular, cerebrovascular and end-organs diseases and disorders in said devices users and devices wearers.

### DESCRIPTION OF THE PREFERRED EMBODIMENTS

The networked smartphones, laptops and mobile digital blood pressure monitor devices (devices and wearables) and desktops—which are commonly and inexpensively available—albeit their clinical significance, practicality and cost-effectiveness in health and healthcare are heretofore oblivious and unbeknownst to billions of people including physicians and other healthcare—in the stores and online.

The palm-size upper arm digital blood pressure monitor with adjustable blood pressure cuff, wrist (forearm) digital blood pressure measurement cuff and smart watch digital blood pressure tracker are the most versatile. They can be improved to be equiped withe miniature circuitries for 24/365 automatically, manually, and regularly monitoring, transmitting and sharing, via cable and wireless, the devices users' and devices wearers' systolic blood pressures (SBP), diastolic, blood pressures (DBP) and heart rates (HR), correlating with and measuring the adaptable cardiovascular hemodynamics, being created and controlled by individual medical prescribed dosages of antihypertensive medications therapies of therapeutic potencies ingested (in) by the devices users and devices wearers.

Similarly, in particular, the networked smartphones and laptops and even networked desktops can serve and collaborate with said watches and cuffs to serve and achieve same function. The networked servers networking said devices and wearables and desktops.

In other words, devices and wearables can be programmed and set by the users and wears, at anytime such as, but not limited to, every ½ hour, 1 hour, ¼ day and any circumstances and settings such as, but not limited to, sitting, eating, working, running, swimming and sleeping to 24/365 automatically, manually, and regularly monitoring, transmitting and sharing the devices users' and devices wearers' systolic blood pressures (SBP), diastolic blood pressures (DBP) and heart rates (HR).

The mobile digital blood pressure monitor devices-transmitted and shared relational devices users' and devices wearers' SBP, DBP and HR in the devices users and devices wearers ingesting individual medical prescribed dosages of antihypertensive medications therapies in the compilations of relational data, which are used to create individual interactive and adaptable digital comparative graphs and, consequently, individual digital comparative composite of predominant and prevailing graphs.

The networked servers networking said devices and wearables and desktops comprised the abiotic intelligence comprising processor units attaching to the storage memories for storing an innovative computer programs including a commercial comparative graphs generator for creating and storing compilations of relational data, in and shared among the storage memories, and in individual interactive and adaptable digital comparative graphs, in and among viewing screens and touchscreens.

The plurality of interactive digital scrolls creating relational antihypertensive medications, dosages of antihypertensive medications, medical prescribed dosages of antihypertensive medications therapies, medical prescribed dosages of antihypertensive medications therapies of therapeutic potencies, the devices users' and devices wearers' SBP, DBP and HR, demographics, volumes of daily oral fluid intakes, physical activities and exertions, and diseases and disorders in the compilations of relational data comprising:
relational the mobile digital blood pressure monitor devices-transmitted and shared relational devices users' and devices wearers' SBP, DBP and HR in the devices users and devices wearers ingesting individual medical prescribed dosages of antihypertensive medications therapies; and relational data of the digital comparative graphs.

$$Normotensive SBP / DBP = 125 / 75 mmHg$$

$$Hypertensive SBP / DBP = > 125 g / 75 mmHg$$

Half of the disease burden occurs in people with hypertensive SBP/DBP>or equal to 140/90 mm Hg (Systemic hypertension: Mechanisms and diagnosis. Braunwald's Heart Disease. 11th ed. 2019 Vol 1 page 910)

The abiotic intelligence getting said relational data in said compilations of relational data to create and adapt individual interactive and adaptable digital comparative graphs comprising:
individual highlighted real-time graphs of hypertensive SBP and DBP, correlating with and measuring the adaptable cardiovascular hemodynamics, of individual devices users and devices wearers taking individual medical prescribed dosages of antihypertensive medications,
individual digital comparative composite of predominant and prevailing graphs of hypertensive SBP and DBP, correlating with and measuring the adaptable cardiovascular hemodynamics, of all devices users and devices wearers ingesting the same individual medical prescribed dosages of antihypertensive medications; and
individual highlighted real-time normotensive SBP and DBP correlating with and measuring the adaptable cardiovascular hemodynamics of individual devices users and devices wearers ingesting individual medical prescribed dosages of antihypertensive medications therapies of therapeutic potencies, superimposing the comparative composite of predominant and prevailing graphs of normotensive SBP and DBP, correlating with and measuring the adaptable cardiovascular hemodynamics of all devices users and devices wearers ingesting the same individual medical prescribed dosages of antihypertensive medications therapies of therapeutic potencies.

The abiotic intelligence creating individual interactive and adaptable digital comparative graphs by instructing the processor units:
scanning and getting individual medical prescribed dosages of antihypertensive medications relating to individual devices users' and devices wearers' hypertensive SBP and DBP in the compilations of relational data,
creating individual interactive and adaptable real-time digital graphs of hypertensive SBP and DBP of individual devices users and devices wearers ingesting individual medical prescribed dosages of antihypertensive medications, in and among viewing screens and touchscreens;
scanning and getting the same individual medical prescribed dosages of antihypertensive medications relating to all devices users' and devices wearers' hypertensive SBP and DBP in the compilations of relational data, and
creating individual interactive and adaptable digital comparative composite of graphs of hypertensive SBP and DBP of all devices users and devices wearers ingesting the same individual medical prescribed dosages of antihypertensive medications in and among viewing screens and touchscreens;
scanning and getting individual medical prescribed dosages of antihypertensive medications therapies of therapeutic potencies relating to individual devices users' and devices wearers' normotensive SBP and DBP and all devices users' and devices wearers' normotensive SBP and DBP, in the compilations of relational data, and
creating individual highlighted real-time normotensive SBP and DBP of individual devices users and devices wearers, ingesting the same individual medical prescribed dosages of antihypertensive medications therapies of therapeutic potencies, superimposing the digital comparative composite of predominant and prevailing graphs of normotensive SBP and DBP of all devices users and devices wearers ingesting the same individual medical prescribed dosages of antihypertensive medications therapies of therapeutic potencies, in and among viewing screens and touchscreens, whereby
the digital comparative composites of predominant and prevailing graphs of normotensive SBP and DBP confirming individual medical prescribed dosages of antihypertensive medications of therapeutic potencies in devices users and devices wearers.

The aforementioned 14 pharmaceutical classes comprising about 95 different antihypertensive medications:
diuretics: thiazide-type diuretics, loop potassium-sparing diuretics, other potassium-sparing diuretics
beta blockers: standard beta blockers, vasodilating beta blockers,
calcium channel blockers; dihydropyridines, nondihydropyridines,
angiotensin-converting enzyme inhibitors,
angiotensin receptor blockers, direct renin inhibitor,
nondihydropyridines;
alpha blockers;
central sympatholytics
direct vasodilators;
nitrates; isosorbids dinitrate, isosorbide 5-mononitrate
fixed-dose combinations of said drugs.

Said 95 different antihypertensive medications such as, but not limited to, lisinopril, amlodipine, felodipine, isosorbide monnitrate, isosorbide dinitrate, Coreq, diltiazem, verapamil, acebutolol, amiloride, furosemide, HCTZ, eplerenone, doxazosin, clonidine, minoxidil, Aliskiren/HCTZ.

Although various preferred embodiments of the present invention have been described, it will be appreciated by and obvious to those skilled in the art and science that adaptations, variations and derivatives of said embodiments are and will be made and achieved without departing from the spirit and scope of the present invention.

1. The abiotic intelligence-created networked communication devices for creating individual interactive and adaptable digital comparative graphs of the devices users' and devices wearers' synchronized and controlled systolic blood pressures (SBP), diastolic blood pressures (DBP) and heart rates (HR), correlating with and measuring the adaptable cardiovascular hemodynamics, being created and controlled by individual medical prescribed dosages of antihypertensive medications therapies of therapeutic potencies in the devices users and devices wearers, and said interactive and adaptable digital comparative graphs for creating and confirming individual medical prescribed dosages of antihypertensive medications of therapeutic potencies for decreasing the risks of and preventing cardiovascular, cerebrovascular and end-organs diseases and disorders in said devices users and devices wearers, collectively known as the abiotic intelligence-rendered blood pressures (AIR Blood Pressures), comprising:
networked smartphones, laptops and mobile digital blood pressure monitor devices and desktops, collectively known as devices and wearables, and servers, wherein the devices and wearables comprised the abiotic intelligence and cable and wireless communication and transmission technologies for sharing and storing the relational data in the compilations of relational data in and share among the storage memories, viewing screens and touchscreens, cameras and microphones;
the abiotic intelligence comprising processor units attaching to the storage memories for storing an innovative computer programs including a commercial comparative graphs generator for creating and storing compilations of relational data, in and shared among the storage memories, and in individual interactive and adaptable digital comparative graphs, in and among viewing screens and touchscreens;
a plurality of interactive digital scrolls in and among the viewing screens and touchscreens;
the antihypertensive medications related to the processor units for networking the networked communication devices in the adaptable compilations of relations in and shared among the storage memories.

2. The AIR Blood Pressures according to claim 1 wherein the devices and wearables comprising:
mobile digital blood pressure monitor devices for 24/365 automatically, manually and regularly monitoring, transmitting and sharing, by cable and wireless technologies, the devices users' and devices wearers' SBP, DBP and HR, correlating with and measuring the adaptable cardiovascular hemodynamics and the synchronized cardiovascular hemodynamics in the devices users and devices wearers ingesting individual medical prescribed dosages of antihypertensive medications therapies, as relational data in the compilations of relational data in and shared among the storage memories and in the interactive and adaptable digital comparative graphs, in and among viewing screens and touchscreens.

3. The AIR Blood Pressures according to claim 1 wherein the mobile digital blood pressure monitor devices comprising:
upper arm digital blood pressure monitor with adjustable blood pressure cuff;
forearm digital blood pressure monitor with adjustable blood pressure cuff;
wrist (forearm) digital blood pressure measurement cuff;
smart watch digital blood pressure tracker; and
the networked smartphones for receiving said devices users' and said devices wearers' SBP, DBP and HR;
the networked laptops for receiving said devices users' and said devices wearers' SBP, DBP and HR;
the networked desktops for receiving said devices users' and said devices wearers' SBP, DBP and HR; and
said cuffs and smart watch for wirelessly connecting to the networked smartphones, laptops, desktops and networked servers.

4. The AIR Blood Pressures according to claim 1 wherein the plurality of interactive digital scrolls, comprising:
a plurality of interactive digital scrolls comprising interactive lists of interactive and selectable dosages of antihypertensive medications, relating the processor units, in viewing screens and touchscreens;
a plurality of interactive digital scrolls comprising interactive lists of interactive and selectable medical prescribed dosages of antihypertensive medications therapies, relating the processor units, in viewing screens and touchscreens;
a plurality of interactive digital scrolls comprising interactive lists of interactive and selectable devices users' and devices wearers' demographics and genetics, volumes of daily oral fluid intakes, physical activities and exertions, and diseases and disorders, relating the processor units; and
a plurality of interactive digital scrolls for inserting and adapting the devices users' and devices wearers' SBP and DBP in the compilations of relational data and individual interactive and adaptable digital comparative graphs;

5. The AIR Blood Pressures according to claim 1 wherein the compilations of relational data comprising the compilations of relational:
the plurality of interactive digital scrolls-created relational antihypertensive medications, dosages of antihypertensive medications, medical prescribed dosages of antihypertensive medications therapies, medical prescribed dosages of antihypertensive medications therapies of therapeutic potencies, the devices users' and devices wearers' SBP, DBP and HR, demographics, volumes of daily oral fluid intakes, physical activities and exertions, and diseases and disorders;
the mobile digital blood pressure monitor devices-transmitted and shared relational devices users' and devices wearers' SBP, DBP and HR in the devices users and devices wearers ingesting individual medical prescribed dosages of antihypertensive medications therapies; and
relational data of the digital comparative graphs.

6. The AIR Blood Pressures according to claim 1 wherein individual interactive and adaptable digital comparative graphs comprising:
individual highlighted real-time graphs of hypertensive SBP and DBP, correlating with and measuring the adaptable cardiovascular hemodynamics, of individual devices users and devices wearers taking individual medical prescribed dosages of antihypertensive medications,
individual digital comparative composite of predominant and prevailing graphs of hypertensive SBP and DBP, correlating with and measuring the adaptable cardiovascular hemodynamics, of all devices users and devices wearers ingesting the same individual medical prescribed dosages of antihypertensive medications; and
individual highlighted real-time normotensive SBP and DBP correlating with and measuring the adaptable cardiovascular hemodynamics of individual devices users and devices wearers ingesting individual medical prescribed dosages of antihypertensive medications therapies of therapeutic potencies, superimposing the comparative composite of predominant and prevailing graphs of normotensive SBP and DBP, correlating with and measuring the adaptable cardiovascular hemodynamics of all devices users and devices wearers ingesting the same individual medical prescribed dosages of antihypertensive medications therapies of therapeutic potencies.

7. The AIR Blood Pressures according to claim 1 wherein the interactive and adaptable digital comparative graphs for creating and confirming individual medical prescribed dosages of antihypertensive medications of therapeutic potencies, comprising:
the comparative composite of predominant and prevailing graphs of normotensive SBP and DBP of all devices users and devices wearers ingesting the same individual medical prescribed dosages of antihypertensive medications therapies of therapeutic potencies

8. The AIR Blood Pressures according to claim 1 wherein individual medical prescribed dosages of antihypertensive medications therapies for creating the adaptable cardiovascular hemodynamics, comprising individual medical prescribed dosages of antihypertensive medications in and among the following pharmaceutical classes:
diuretics;
beta blockers;
vasodilating beta blockers;
calcium channel blockers;
nondihydropyridines;
angiotensin-converting enzyme inhibitors;
angiotensin receptor blockers;
direct renin inhibitor;
alpha blockers;
central sympatholytics
direct vasodilators;
nitrates; and
fixed-dose combinations of said drugs.

9. The AIR Blood Pressures according to claim 1 wherein individual medical prescribed dosages of antihypertensive medications therapies of therapeutic potencies for creating synchronized and controlled cardiovascular hemodynamics, comprising individual medical prescribed dosages of antihypertensive medications of therapeutic potencies in and among the following pharmaceutical classes:
diuretics;
beta blockers;
vasodilating beta blockers;
calcium channel blockers;
nondihydropyridines;
angiotensin-converting enzyme inhibitors;
angiotensin receptor blockers;
direct renin inhibitor;
alpha blockers;
central sympatholytics
direct vasodilators;
nitrates; and
fixed-dose combinations of said drugs.

10. In AIR Blood Pressures, the method of the abiotic intelligence-created networked communication devices creating individual interactive and adaptable digital comparative graphs of the devices users' and devices wearers' synchronized and controlled SBP, DBP and HR, correlating with and measuring the adaptable cardiovascular hemodynamics, being created and controlled by individual medical prescribed dosages of antihypertensive medications therapies of therapeutic potencies, in and among viewing screens and touchscreens, and said
interactive and adaptable digital comparative graphs creating and confirming individual medical prescribed dosages of antihypertensive medications of therapeutic potencies ingested by the devices users and devices wearers, thereby decreasing the risks of and preventing cardiovascular, cerebrovascular and damages of the organs, diseases and disorders in the devices users and devices wearers, the method comprising:
the abiotic intelligence comprising the processor units attaching to the storage memories storing the compilations of relational data and the innovative computer programs including the commercial comparative graphs generator to instructing the processor units to:
creating a plurality of interactive digital scrolls creating, compiling and adapting the relational data in the compilation of the relational data, whereby the antihypertensive medications related to the processor units networking the networked communication devices, in the adaptable compilations of relational data in and shared among the storage memories;
effecting the mobile digital blood pressure monitor devices 24/365 automatically, manually, and regularly monitoring, transmitting, via cable and wireless, the devices users' and devices wearers' SBP, DBP and HR, correlating with and measuring the adaptable cardiovascular hemodynamics, being created by individual medical prescribed dosages of antihypertensive medications therapies ingested by the devices users and devices wearers;

11. The method according to claim 10 whereby the mobile digital blood pressure monitor devices for 24/365 automatically, manually and regularly monitoring, transmitting and sharing, by cable and wireless technologies, the devices users' and devices wearers' SBP, DBP and HR, correlating with and measuring the adaptable cardiovascular hemodynamics and the synchronized cardiovascular hemodynamics in the devices users and devices wearers ingesting individual medical prescribed dosages of antihypertensive medications therapies, as relational data in the compilations of relational data in and shared among the storage memories and in the interactive and adaptable digital comparative graphs, in and among viewing screens and touchscreens.

12. The method according to claim 10 whereby the processor units creating a plurality of interactive digital scrolls creating, compiling and adapting the relational data in he compilation of the relational data, comprising the process of the processor units:
creating a plurality of interactive digital scrolls comprising interactive lists of interactive and selectable dosages of antihypertensive medications, relating the processor units, in viewing screens and touchscreens;
creating a plurality of interactive digital scrolls comprising interactive lists of interactive and selectable medical prescribed dosages of antihypertensive medications therapies, relating the processor units, in viewing screens and touchscreens;
creating a plurality of interactive digital scrolls comprising interactive lists of interactive and selectable devices users' and devices wearers' demographics and genetics, volumes of daily oral fluid intakes, physical activities and exertions, and diseases and disorders, relating the processor units;
creating a plurality of interactive digital scrolls for inserting and adapting the devices users' and devices wearers' SBP and DBP in the compilations of relational data and individual interactive and adaptable digital comparative graphs;
compiling and relating said plurality of interactive digital scrolls-created, related and adapted relational data in the adaptable compilations of relational data, in and shared among the storage memories; and
networking the networked communication devices comprising the processor units relating to individual dosages of antihypertensive medications, in the adaptable compilations of relations of relational data in and shared among the storage memories; and
networking the networked communication devices comprising the processor units relating to individual medical prescribed dosages of antihypertensive medications therapies, in the adaptable compilations of relations of relational data in and shared among the storage memories.

13. The process of the plurality of interactive digital scrolls inserting and adapting the devices users' and devices wearers' SBP and DBP in the compilations of relational data and individual interactive and adaptable digital comparative graphs according to claim 12, comprising the process of the the processor units;
relating to individual dosages of antihypertensive medications relating to
SBP and DBP of individual devices users and devices wearers demographics, and
the adapted SBP and DBP of individual devices users and devices wearers demographics, in the adaptable compilations relational data in and shared among the storage memories;
scanning and getting individual medical prescribed dosages of antihypertensive medications relating to individual devices users' and devices wearers' SBP and DBP and adapted SBP and DBP in the compilations of relational data, and
creating individual interactive and adaptable real-time digital graphs of adapted SBP and DBP in lieu of SBP and DBP of the same individual devices users and devices wearers, in and among viewing screens and touchscreens.

14. The method according to claim 10 whereby the processor units creating individual interactive and adaptable digital comparative graphs, comprising the processes of the processor units:
scanning and getting individual medical prescribed dosages of antihypertensive medications relating to individual devices users' and devices wearers' hypertensive SBP and DBP in the compilations of relational data,
creating individual interactive and adaptable real-time digital graphs of hypertensive SBP and DBP of individual devices users and devices wearers ingesting individual medical prescribed dosages of antihypertensive medications, in and among viewing screens and touchscreens;
scanning and getting the same individual medical prescribed dosages of antihypertensive medications relating to all devices users' and devices wearers' hypertensive SBP and DBP in the compilations of relational data, and
creating individual interactive and adaptable digital comparative composite of graphs of hypertensive SBP and DBP of all devices users and devices wearers ingesting the same individual medical prescribed dosages of antihypertensive medications in and among viewing screens and touchscreens;
scanning and getting individual medical prescribed dosages of antihypertensive medications therapies of therapeutic potencies relating to individual devices users' and devices wearers' normotensive SBP and DBP and all devices users' and devices wearers' normotensive SBP and DBP, in the compilations of relational data, and
creating individual highlighted real-time normotensive SBP and DBP of individual devices users and devices wearers, ingesting the same individual medical prescribed dosages of antihypertensive medications therapies of therapeutic potencies, superimposing the digital comparative composite of predominant and prevailing graphs of normotensive SBP and DBP of all devices users and devices wearers ingesting the same individual medical prescribed dosages of antihypertensive medications therapies of therapeutic potencies, in and among viewing screens and touchscreens, whereby
the digital comparative composites of predominant and prevailing graphs of normotensive SBP and DBP confirming individual medical prescribed dosages of antihypertensive medications of therapeutic potencies in devices users and devices wearers.
